(19) United States
(12) Patent Application Publication
(10) Pub. No.: US 2016/0002598 A1
(43) Pub. Date: Jan. 7, 2016

(54) DEVICE AND METHODS FOR PLATELET LYSIS OR ACTIVATION

(71) Applicants: Chirstopher CENTENO, Broomfield, CO (US); Brian J. LEACH, Broomfield, CO (US); Ryan DREGALLA, Broomfield, CO (US); Patrick REISCHLING, Broomfield, CO (US); Nicolette LYONS, Broomfield, CO (US); REGENERATIVE SCIENCES, LLC, Broomfield, CO (US)

(72) Inventors: Christopher J. Centeno, Boulder, CO (US); Brian J. Leach, Denver, CO (US); Ryan Dregalla, Ft. Collins, CO (US); Patrick Reischling, Broomfield, CO (US); Nicolette Lyons, Westminster, CO (US)

(73) Assignee: Regenerative Science, LLC, Broomfield, CO (US)

(21) Appl. No.: 14/761,828

(22) PCT Filed: Jan. 28, 2014
(86) PCT No.: PCT/US14/13351

§ 371 Date: Jul. 17, 2015

Related U.S. Application Data
(60) Provisional application No. 61/849,444, filed on Jan. 28, 2013.
(60) Provisional application No. 61/824,090, filed on May 16, 2013.

(51) Int. Cl. C12N 5/078; C12M 3/04; C12M 1/00; C12M 1/42; A61K 35/19; C12N 13/00

(52) U.S. Cl. CPC C12N 5/0644; A61K 35/19; C12N 13/00; C12M 47/06; C12M 29/00; C12M 35/04; C12M 35/00; C12M 23/28; C12M 27/10; C12N 2523/00; C12N 2521/10

(57) ABSTRACT

Device, system and method embodiments are disclosed herein which provide for the production of a modified autologous platelet solution at the patient bedside for contemporaneous reinjection to the patient. In certain embodiments all of the steps including, but not limited to blood draw, platelet lysis, solution preparation and reinjection to a patient may be accomplished in a single office or clinic visit without relocating the patient. Accordingly, the apparatus, devices and systems disclosed herein generally include a substantially stand-alone machine, device or system which is configured to accept a platelet containing solution, induce lysis of one or more platelet bodies within the platelet containing solution and provide the resulting modified platelet solution in a manner suitable for injection into the patient.

### TECHNICAL FIELD

The embodiments disclosed herein are directed toward a device, system and methods for platelet lysis or activation. Embodiments are more particularly directed toward systems and methods for platelet lysis or activation which may be implemented at a patient's bedside during a single treatment session.

### BACKGROUND

Platelets are small, disc shaped non-nucleated cell fragments which circulate in the blood of mammals. Platelets are a natural source of growth factors including but not limited to platelet-derived growth factor (PDGF), transforming growth factor beta (TGF-β), fibroblast growth factor, insulin-like growth factor epidermal growth factor, vascular endothelial growth factor and others. In addition to the foregoing factors, platelets also include a granules, cytokines, proteins, cellular components, mRNA, ribosomal RNA, transfer RNA, DNA, small molecules including chemicals, hormones and signaling molecules. The foregoing factors and other platelet contents are referred to herein collectively as “therapeutic platelet contents.” Therapeutic platelet contents have been shown to play a significant role in the repair and generation of injured or damaged biological tissue including but not limited to human connective tissues. Local application of various platelet-derived therapeutic platelet contents in increased concentration by the administration of a solution enriched with the content of autologous platelets is a known technique to promote wound healing.

Many methods are known to cause or induce lysis or the disruption of a cellular or cell fragment membrane for the purpose of releasing the contents of the cell or platelet into solution. Typical methods may be grouped into six categories; Optical, mechanical, acoustic, electrical, chemical, and thermal. One or more of the foregoing methods can be employed for batch lysis of a platelet containing solution. Alternatively, lysis methods have been be applied to a single cell for analysis of the contents.

Known methods of platelet lysis require extensive capital equipment, specialized disposable consumables, and complex techniques. Additionally, the time required to create modified platelet solutions using known techniques are sufficiently lengthy that it is not reasonable to begin with freshly drawn or pre-processed patient blood and create an injectable modified platelet solution within the time frame of a single office visit. Furthermore, a medical provider is very unlikely to have access to the clean room and laboratory equipment necessary to produce and process a suitable modified platelet solution on site. These difficulties have restricted the adoption of autologous platelet lysate therapies to specialized labs at high cost and prohibited the use of autologous platelet lysate therapies in a normal clinical setting. The embodiments disclosed herein are directed toward overcoming one or more of the problems discussed above.

### SUMMARY OF THE EMBODIMENTS

Device, system and method embodiments are disclosed herein which provide for the production of a modified autologous platelet solution at a patient bedside for contemporaneous reinjection to the patient. In certain embodiments all of the steps including, but not limited to blood draw, platelet lysis/activation, solution preparation and reinjection to a patient may be accomplished in a single office or clinic visit without relocating the patient.

Accordingly, the apparatus, devices and systems disclosed herein generally include a substantially stand-alone machine, device or system which is configured to accept a platelet containing solution, induce lysis or activation of a quantity of platelet bodies within the platelet containing solution and provide the resulting modified solution in a manner suitable for injection into the patient. The described embodiments therefore provide for the creation of an injectable modified solution without the requirement of additional laboratory-based equipment, aside from the described devices and associated consumable or disposable apparatus or parts.

One embodiment disclosed herein is a device having a housing. An input port is provided through the housing which allows for the input of a platelet containing solution into the device. From the input port, a platelet containing solution is flowed, transported or otherwise placed into a lysis/activation chamber also positioned within the device housing. Within the lysis/activation chamber, one or more platelets of the platelet containing solution are caused to undergo lysis or activation as described below. Thus a modified solution is formed within the lysis/activation chamber. An outlet port is provided from the housing in fluid communication with the lysis/activation chamber which provides for the modified solution to be removed from the outlet port.

Lysis or activation of platelets within the lysis/activation chamber can be caused by several disclosed techniques or any combination of techniques. For example, lysis and/or activation of platelets may be caused by subjecting the platelet containing solution to one or more whole or partial freeze/thaw cycles. Therefore, the platelet lysis/activation chamber may be in thermal contact with a heating and cooling module also maintained within the device housing. The heating and cooling module may be utilize one of several techniques to heat and cool the platelet containing solution including but not limited to contacting the lysis/activation chamber with a gas, liquid or other heating and cooling medium, applying conventional refrigeration or heating cycles or other means. In one non-limiting representative example, the lysis/activation chamber may comprise a length of disposable sterile tubing which is contacted with a heating or cooling medium within the housing.

Alternative embodiments of a device or system may include one or more supplemental input ports into the housing and in communication with the lysis/activation chamber. Said supplemental input port or ports may provide for the introduction of a substance into contact with the platelet containing solution to cause or promote lysis or activation of one or more platelets within the lysis/activation chamber. The lysis or activation causing substance may be but is not limited to CaCl₂, an alternative salt, ADP, epinephrine, thrombin, collagen, or von Willebrand factor.

Other alternative apparatus may be provided within the housing to cause or promote platelet lysis or activation. The alternative apparatus may subject the platelet containing solution to processes including but not limited to the application of acoustic energy, subjecting the platelet containing solution to shear stress, subjecting the platelet containing solution to osmotic stress or contacting the platelet containing solution with an activation promoting surface or substance including but not limited to glass or collagen. A device embodiment may include elements providing for any combination of thermal, chemical, mechanical or other platelet lysis or activation steps.

The outlet port of a device embodiment may include a filter. In addition, in certain embodiments the lysis/activation chamber may be in communication with a vacuum source providing for the removal of a fluid such as plasma from the platelet containing solution prior to or after the performance of selected lysis or activation steps. Furthermore, the apparatus may include a rotation device coupled to the lysis/activation chamber and providing for the rotation of the lysis/activation chamber within the housing, causing the concentration of platelets within the platelet containing solution before or after selected lysis/activation steps.

Alternative embodiments include methods of preparing a modified solution from a platelet containing solution utilizing one or more embodiments of device as disclosed herein. Methods include the steps of introducing a platelet containing solution into the input port of a device through a housing. Method embodiments also include a process for flowing or otherwise transporting the platelet containing solution to a lysis/activation chamber within the housing and causing the lysis or activation of platelets within the platelet containing solution in the lysis/activation chamber. Therefore, method embodiments result in a modified solution prepared within a stand-alone device which is suitable for use at a patient's bedside or in a clinic. Method embodiments may further comprise reinjection of the modified solution into a patient.

Method embodiments may feature any combination of techniques to cause whole or partial lysis and/or activation of platelets within the lysis/activation chamber. Lysis/activation techniques include but are not limited to heating and cooling the solution to cause one or more freeze/thaw cycles, desiccating the solution, subjecting the solution to shear stress, subjecting the solution to acoustic energy, mixing the solution with one or more lysis/activation causing agents, flowing the solution over one or more lysis or activation causing surfaces, subjecting the solution to osmotic stress or other means.

### DESCRIPTION OF THE DRAWINGS

FIG. 1 is a schematic diagram of a device and system as disclosed herein.

FIG. 2 is a flowchart representation of a method as described herein.

FIG. 3A-3I a schematic representations of an alternative device and method embodiment.

FIG. 4 is a flowchart representation of an alternative method embodiment.

FIG. 5 is a graph comparison showing the percentage increase of selected platelet lysis or activation parameters after multiple freeze/thaw cycles compared to a similar sample subjected to only one freeze/thaw cycle at selected freeze/thaw temperature ranges.

FIG. 6 is a graph comparison showing the percentage increase or decrease of selected platelet lysis or activation parameters after multiple freeze/thaw cycles utilizing isopropyl alcohol as the cooling medium compared to a similar sample subjected to similar freeze/thaw cycles utilizing air as the cooling medium.

FIG. 7 is a graph comparison showing the percentage increase of selected platelet lysis or activation parameters comparing a sample subjected to multiple freeze/thaw cycles and shear stress plus the addition of CaCl₂ as a lysis/activation agent with a similar sample subjected to multiple freeze/thaw and shear stress cycles as the sole lysis/activation steps.

FIG. 8 is a graph comparison showing the percentage increase of selected platelet lysis or activation parameters comparing a sample placed under osmotic stress with a hypotonic solution and a similar sample not placed under osmotic stress.

FIG. 9 is a graph comparison showing the percentage increase of selected platelet lysis or activation parameters after one or more freeze/thaw cycles after platelet isolation by centrifugation compared to a similar sample subjected to one or more freeze/thaw cycles without platelet isolation by centrifugation.

FIG. 10 is a graph comparison showing the percentage increase of selected platelet lysis or activation parameters after a representative combination process including multiple freeze thaw cycles with the platelets centrifuged out of solution and exposed to a selected concentration of calcium chloride and water providing for osmotic stress compared with a similar sample subjected only to one or more freeze/thaw cycles.

### DETAILED DESCRIPTION

Unless otherwise indicated, all numbers expressing quantities of ingredients, dimensions reaction conditions and so forth used in the specification and claims are to be understood as being modified in all instances by the term “about”.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

The various device, system and method embodiments disclosed herein provide for the production of a modified autologous platelet solution at the patient bedside for contemporaneous reinjection to the patient. In certain embodiments all of the steps including, but not limited to blood draw, platelet lysis and/or platelet activation, solution preparation and reinjection to a patient may be accomplished in a single office or clinic visit without relocating the patient. The terms “platelet lysis” are defined herein as a process or method that results in the rupture of a platelet cell membrane, thereby releasing therapeutic contents from the platelet. The terms “platelet activation” are defined herein as a process that triggers a series of events that control platelet aggregation, adherence and the release or specific proteins and growth factors to promote wound healing. Platelet activation can occur in the blood stream, for example in response to a wound. The platelet activation referred to herein occurs outside of the human body and causes the release of therapeutic contents from the platelet without necessarily causing the rupture of a platelet cell membrane.

Accordingly, the apparatus, devices and systems disclosed herein generally include a unified machine, device or system which is configured to accept a platelet containing solution, induce lysis and/or activation of one or more platelet bodies within the platelet containing solution and provide the resulting modified platelet solution in a manner suitable for injection into the patient. The described embodiments therefore provide for the creation of an injectable modified platelet solution without the requirement of additional laboratory-based equipment, aside from the described devices and associated consumable or disposable apparatus.

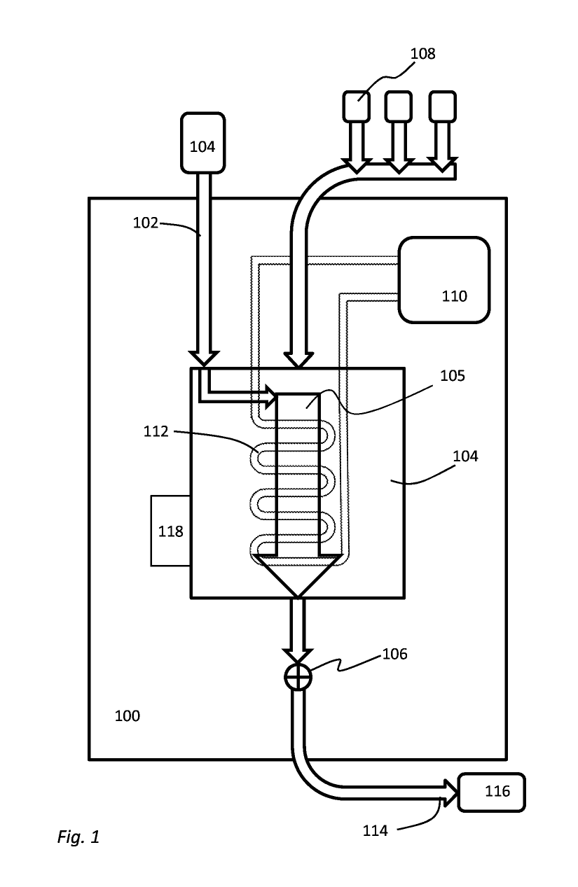

In one device and method embodiment, a modified platelet solution is created by using thermal energy to freeze and subsequently thaw a platelet containing solution thereby causing the platelet bodies to release their therapeutic contents through lysis and/or activation. For example, FIG. 1 schematically illustrates a system embodiment 100 that features the use of a heating and cooling apparatus to freeze some or all of the platelet containing solution to induce lysis and or activation of a quantity of platelets in the platelet containing solution. In the FIG. 1 system 100, a syringe, sterile bag or other suitable container 104 containing a patient's autologous platelet containing solution is connected to the system 100 at an input port 102. The patient's autologous platelet containing solution is derived from the patient's blood. The platelet containing solution may have been preprocessed to concentrate the platelets, diluted, mixed with other fluids or otherwise modified. Typically, the platelet containing solution will be pre-processed and prepared from blood drawn from the patient at the commencement of a treatment session. Alternatively, the platelet containing solution may be prepared from blood drawn from the patient at an earlier date and stored before or after pre-processing.

The platelet containing solution is transported through the system 100 to and through a lysis/activation chamber 104. The platelet containing solution may be transported in a fluid pathway 105 having any selected shape, volume or configuration. In the particular system embodiment of FIG. 1, the lysis/activation chamber 104 is implemented as a thermal lysis/activation chamber. Alternative lysis/activation chambers which rely on alternative lysis/activation methods are described in detail below. The lysis/activation chamber 104 and the fluid pathway 105 associated therewith can be of any suitable shape or configuration and have any selected volumes. Typically, the platelet containing solution is transported in a fluid pathway 105 comprising, at least in part, sterile tubing which may, as detailed below, be disposable sterile tubing. Valves, meters, pumps, gates, storage reservoirs and other fluid control apparatus may be implemented as required to control the flow of fluids within the system. For example, an exit valve 106 may be utilized to cause the platelet containing solution to remain within the lysis/activation chamber for a specified time.

The system 100 may also include one or more supplemental fluid or composition ports 108 which optionally may be used to accept typically disposable containers or other input configurations or quantities of consumable adjuncts, chemicals, additives, solvents or other substances used during the platelet lysis and/or activation process. The supplemental ports 108 are placed in fluid communication the lysis/activation chamber 104 and/or fluid pathway 105 using tubing, pipes, solid material conveyor systems or other material handling apparatus. For example, one or more valves, pumps or material conveyors with or without associated digital control apparatus may be implemented to control the timing and mixing rate of any desired secondary substances with the platelet containing solution within the fluid pathway 105 and/or lysis/activation chamber 104.

The amount of platelet containing solution introduced into the system 100 may be predetermined such that the volume of the fluid pathway 105 within the lysis/activation chamber 104 is occupied with the entirety of the platelet containing solution volume introduced into the system. Thus, the system 100 may be utilized to process a defined batch of platelet containing solution prior to reinjection into the patient or other therapeutic usage. Alternatively, the system may be configured to continuously process platelet containing solution as it is fed into the system 100 and reinjected into the patient.

As noted above, the particular implementation illustrated in FIG. 1 includes a thermal lysis/activation chamber 104. Accordingly, the system 100 includes a heating and cooling module 110 in thermal communication with the lysis chamber 104. The heating and cooling module 110 may be implemented with a heat exchange apparatus, conventional electric, gas or other heating and chilling elements of any type or another type of heating and cooling apparatus. The heating and cooling module 110 thermally communicates with the lysis/activation chamber 104 through heat exchange surfaces 112. For example, the heat exchange surfaces 112 may be configured such that surface area contact of the heat exchange surfaces 112 and the thermal lysis/activation chamber 104 are maximized.

The heating and cooling module 110 may be configured to remove heat from the platelet containing solution through the heat exchange surfaces 112 until the platelet containing solution partially or entirely freezes. After this complete or partial phase change occurs, heat may be applied to the thermal lysis chamber 104 with the heating and cooling module 110, for example by reversed operation of a heat exchanger, conventional heaters or other means. Heating the partially or entirely frozen platelet containing solution causes the frozen portions of the platelet containing solution to thaw. One or more complete freeze/thaw cycles may be implemented within the lysis/activation chamber 104. During the one or more freeze/thaw cycles, platelet lysis/activation occurs causing the therapeutic platelet contents to be released into the solution thereby creating a platelet-lysate solution (PL solution) that may or may not contain intact activated platelets. After a final thawing and/or warming of the PL solution, the valve 106 may be opened to allow the PL solution to flow towards an outlet port 114 and into a second container (for example, a syringe) suitable for use as a vehicle providing for the readministration some or all of the PL solution to the patient.

The system 100 may be implemented as a substantially self-contained device with all elements other than the removable containers 104, 108 and 116 housed within a single, possibly portable, housing. Furthermore, all components that are wetted by the platelet containing solution may be implemented with disposable parts that are replaced after each procedure. The use of a disposable tubing kit to implement at least a portion of the fluid pathway 105 serves to ensure the use of a sterile apparatus for each patient.

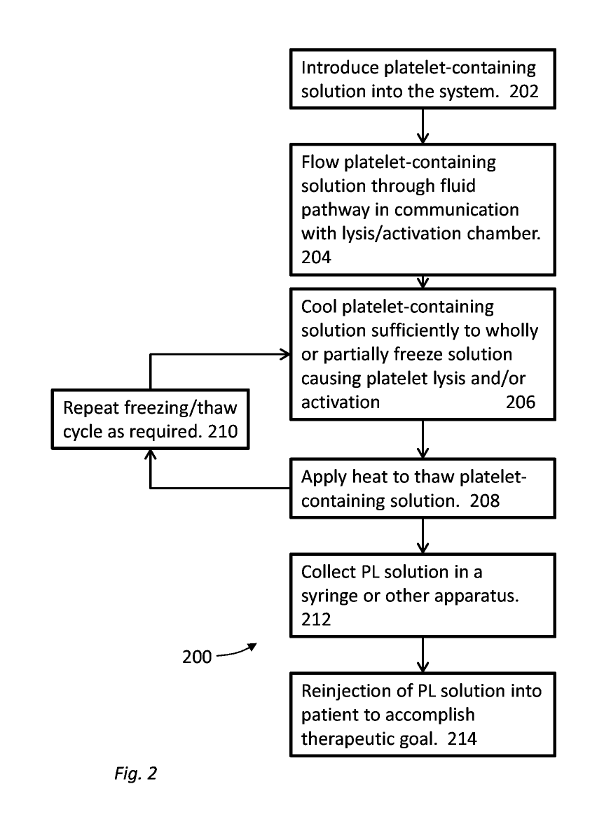

The system of FIG. 1 may be utilized as follows, according to a non-exclusive method 200 as illustrated in FIG. 2. Platelet containing solution derived previously or contemporaneously from a patient's blood is introduced into the system 100 (step 202). The platelet containing solution is routed through a suitable system of conduits into the fluid pathway 105 of a thermal lysis/activation chamber 104 (step 204). As noted above, the lysis/activation chamber 104 provides for the platelet containing solution to be contacted with thermal energy in one or more cooling and/or heating cycles. As noted above, the configuration of the heating and cooling module 110 may be of various configurations relying upon various energy sources and heat exchange methods. For example, in one specific device configuration, a shell and tube system may be incorporated such that the platelet containing solution is first introduced into an inlet plenum and then distributed into one or more tubes. The flow of the platelet containing solution is controlled such that some, or the entire quantity of platelet containing solution, is moved into one or more of said tubes. Once the platelet containing solution is in the heat exchange tubes, flow may be caused to stop.

In this particular embodiment, the tube or tubes are contained within a shell. There is no fluid communication between the tubes and shell. The shell and tube system is configured such that surface area of the tube or tubes with the internal volume of the shell is maximized

A heat exchange fluid is introduced into the shell through a first entry port at a first temperature that is lower than the freezing point of the platelet containing solution. The temperature of the heat exchange fluid may be controlled by the heating and cooling module 110, associated sensors and associated control apparatus. The heat exchange fluid removes heat from the tube or tubes and as a result, from the platelet containing solution contained therein. The heat exchange fluid exits the shell through a second exit port at a second temperature that is higher than the first temperature. Thus, heat is removed from the platelet containing solution until a sufficient temperature drop in the platelet containing solution induces a partial or total liquid to solid phase change in the liquid platelet containing solution (step 206). Formation of ice crystals within the platelet membrane causes volume expansion of the platelet contents thereby imparting mechanical stress on the platelet membrane. The mechanical stress causes rupture of this membrane and release of the therapeutic platelet contents including various factors of interest into the surrounding solution. Additionally as noted below, freezing can cause the activation of platelets that did not undergo lysis

After the platelet containing solution has partially or completely undergone a phase change from liquid to solid, the heat exchange process may be selectively reversed such that the platelet containing solution undergoes a thawing phase change from a solid to liquid (step 208). In one embodiment, a temperature sensor in communication with a digital control system monitors the temperature of the platelet containing solution to determine when certain temperature benchmarks, for example the freezing or thawing point, are reached. In other embodiments, control will be based upon empirical data and modeling of the behavior of the system to pre-program a time for the cycle of platelet containing solution heat removal to ensure that partial or complete phase change of the platelet containing solution has taken place. If desired to achieve sufficient platelet lysis and/or activation, the cycles of freezing and thawing may be repeated as required (step 210).

Alternative methods may be utilized to identify and/or control one or more cycles of partial or complete phase change within the platelet containing solution. Alternative monitoring and control methods include, but are not limited to monitoring one or more parameters including but not limited to the volume change of the platelet containing solution, optical transmittance of the platelet containing solution, acoustic wave transmission within the platelet containing solution, and others.

After one or more of freeze/thaw cycles are performed to ensure lysis and/or activation of a sufficient number of platelet bodies, a final warming or thawing step may be performed. At this point, the platelet containing solution may be referred to as a platelet lysate solution (PL solution) or alternatively referred to as a modified solution containing one or more lysed or activated platelet bodies. The PL solution may then be routed out of the thermal lysis/activation chamber 104 and into a container suitable for reinjection into the patient (steps 212 and 214). As noted above, the system 100 may be configured such that all components wetted by the platelet containing solution or PL solution are designed as an easily replaceable and disposable. In addition, all steps may be performed in a self-contained, possibly portable system located at a physician's office, clinic or hospital room. Thus, the entire treatment method may be implemented while requiring only a single or limited number of office visits from the patient.

The system 100 is not limited to the elements recited specifically above. For example, in an alternative configuration of the thermal lysis/activation chamber 104, the platelet-containing fluid is routed within a fluid pathway 105 including a chamber whose bounding walls are in direct contact with a heating/cooling element or a heat exchanger. The chamber may be configured such that the surface area of the chamber bounding walls in contact with the platelet containing solution fluid is maximized. Additionally, the chamber may be configured such that the surface area of the chamber in contact with the heat exchanger is maximized In this embodiment, the platelet containing solution is routed into the chamber until the platelet containing solution substantially occupies the entirety of the inner volume of the chamber, at which point flow is caused to stop. The heat exchanger or other heating/cooling element removes heat from the platelet containing solution until partial or complete phase change of the platelet containing solution occurs. A cycle of freezing and thawing may proceed as described above until a PL solution is produced and removed from the system for reintroduction by injection or other method into the patient.

Many possible variations of heating and cooling module relying on direct or indirect heat exchange are within the scope of the present disclosure. For example, in one embodiment, the heating and cooling elements are implemented as a closed system with a non-consumable heat transfer medium. Accordingly, the level of heat transfer medium in the system does not diminish throughout the course of multiple uses of the device. Such a heat exchange system may be implemented with compression and expansion chambers exploiting a vapor compression cycle similar to a standard air conditioning or refrigeration system employing known heat transfer mediums such as R-11, R-12, R-114, R-22, R123, R-134a, R-502, R-40, R-764, R-170, or R-290.

In other embodiments of the system, the heat transfer medium may be introduced into the system on an as-needed basis. For example, a disposable container of gas, fluid, or liquefied gas (or a connection to gas or fluid supply) may be used to rapidly remove heat from the platelet containing solution. The temperature drop that occurs when compressed gas occupying a small volume is allowed to expand into a higher volume at a lower pressure may be exploited to remove heat from the platelet containing solution. A suitable cooling gas used in an open ended system may be inert and environmentally safe such that it may be released into the atmosphere without negative consequence. Other less inherently safe gases may require filtration, treatment, or a means of recapture. Suitable heat transfer mediums for an open system include, but are not limited to carbon dioxide, oxygen, helium, hydrogen, nitrogen, and others. The cooling gas or fluid may be released directly into the room, released into building HVAC, or vented outside of the building for direct release into the atmosphere. In open-ended embodiments, the container of heat transfer medium is designed as a consumable product that will be replaced after each procedure or a specified number of procedures depending on need.

In alternative embodiments, a lysis and/or activation method other than temperature-induced phase change may be utilized to accomplish platelet lysis and/or platelet activation. For example, a platelet containing solution may be introduced to a system 100 featuring a lysis/activation chamber 104 implemented as a mixing chamber. In the mixing chamber, platelet containing solution is mixed with a lysis and/or activating inducing compound or substance such as calcium chloride (CaCl), thrombin or others.

The effectiveness of a bedside located system featuring any type of lysis/activation chamber may be enhanced by introducing other agents to cause the platelets to release therapeutic contents into solution. Supplemental methods of causing platelets to release their contents via biological mechanisms are referred to herein as methods of “activating” the platelets. The volume of selected activation agents added to the platelet containing solution may be determined on a patient-to-patient basis. The addition of activating agents causes the platelet bodies to more effectively release the therapeutic content within said platelet bodies. Activating agents include, but are not limited to thrombin, CaCl₂, ADP and epinephrine.

Methods for activating platelets also include exposing platelets to high shear stresses and exposing platelets to glass or collagen coated surfaces. Therefore, additional chambers or fluid pathways may be included within the system 100 or lysis/activation chamber 104 that enhance the activation of platelets. For example, a system may include one chamber providing a freeze thaw stage after which the platelet solution then flows through glass or collagen coated tubing before collection for injection. Similarly, one or more freeze/thaw cycles may be provided in one chamber followed by the subsequent flowing of the platelet solution through a coated tube and back into the freeze/thaw chamber to maximize the amount of lysis and activation with each step repeated as required.

High shear stresses suitable for platelet activation may be obtained by forcing the platelet solution through very small tubing similar in size to a 22 g to 27 g needle. The various methods of causing lysis or platelet activation may be provided in any order. For example, a platelet containing solution may be fed into a system by a high pressure pump feeding a relatively narrow passageway or aperture. Thus, the platelets are subjected to shear stress. The solution may then enter processing subsequent chambers where freeze/thaw cycles are applied or where activating agents such as chloride, thrombin, ADP or epinephrine are mixed with the platelet containing solution. Each separate process could then be repeated as required to maximize platelet lysis and activation.

In alternative embodiments, lysis or activation of the platelet bodies is induced by the application of acoustic energy (sonication) of the platelet containing solution. Sonication refers to the coupling of acoustic energy at a select frequency and amplitude to cause lysis and/or activation within the platelet containing solution. In a sonication embodiment, the platelet containing solution is introduced into the system and routed through the fluid pathway 105 to a lysis chamber implemented as a sonication chamber. In the sonication chamber, ultrasonic vibrations are coupled to the solution from one or more suitable transducers 118 which induce localized high pressure areas that cause cavitation and subsequent shearing of the platelet membranes. Thermal management of the sonication chamber is advantageous so that the platelet containing solution temperature does not rise above a predetermined critical temperature that may denature proteins or otherwise cause irreversible damage to the solution.

In yet another embodiment, lysis/activation of the platelets is induced by osmotic stress caused by introducing the platelet containing solution to a hypotonic solution. Osmotic pressure causes water to enter the platelet body from the surrounding solution. The water will continue to cross the membrane until the platelet membrane is mechanically stressed such that lysis or activation of the platelet occurs. The hypotonic solution is advantageously a material that is safe to be injected, for example water.

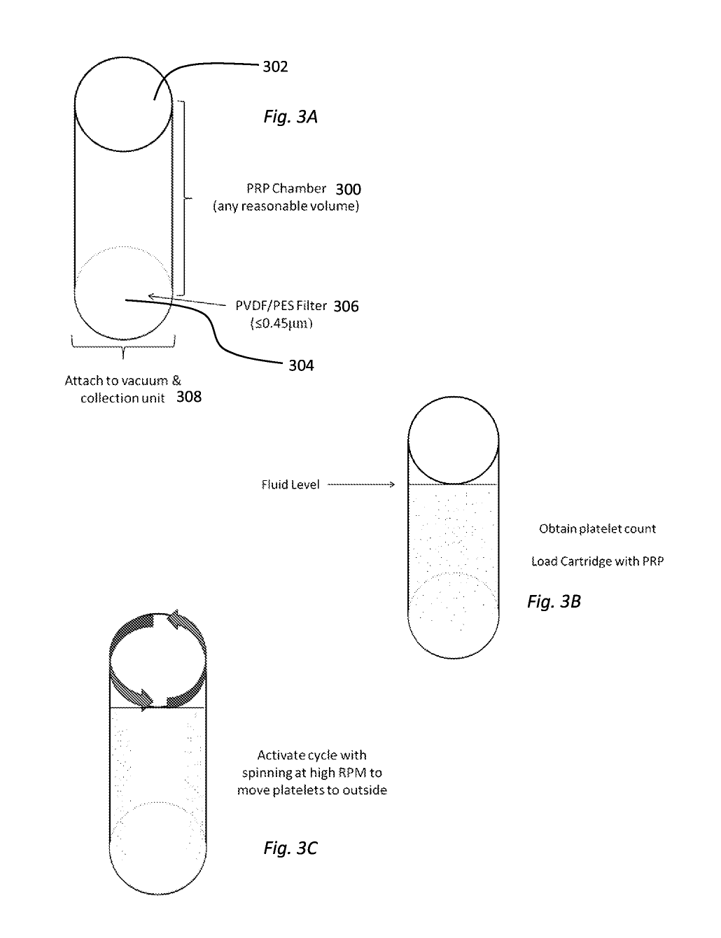

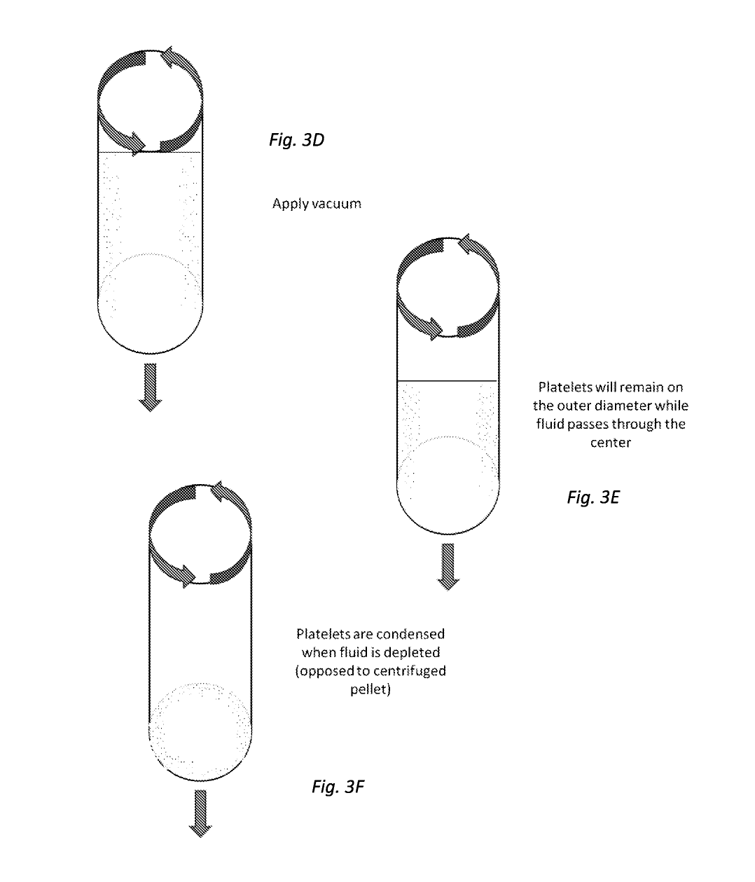

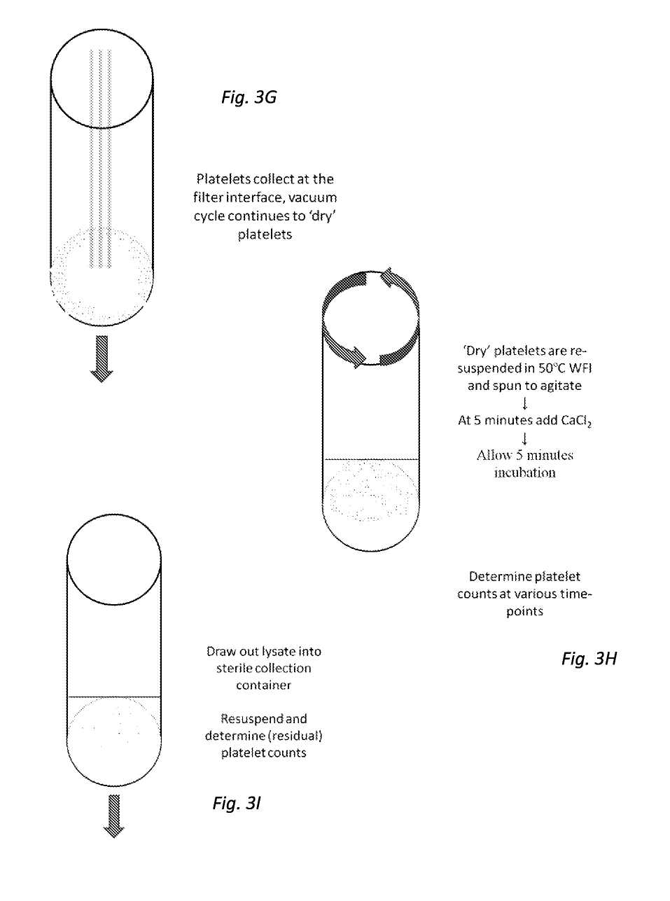

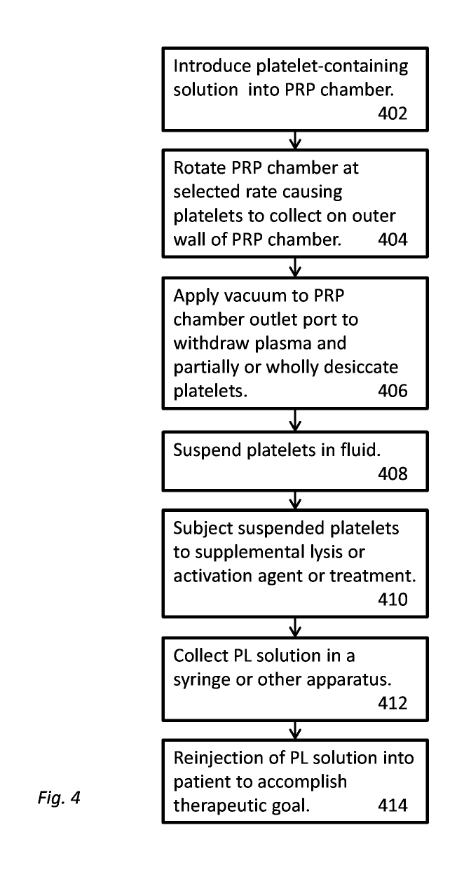

Another alternative device and method which may be utilized to effectively cause platelet lysis and/or activation is schematically illustrated in FIGS. 3A-3I and FIG. 4. The embodiment of FIG. 3 and FIG. 4 may be implemented within a combination lysis/activation chamber 104 and fluid pathway 105 of a device 100 similar to that shown in FIG. 1. Thus, the FIG. 3 and FIG. 4 embodiment may also be implemented in a bedside system which accomplishes all steps within a single device which is located at a treating physician's office, hospital room, clinic or otherwise at a patient bedside.

The apparatus and methods illustrated in FIG. 3 and FIG. 4 may be implemented relatively rapidly and will result in a high quality lysate or activated modified solution. In particular, the lysis/activation method and apparatus illustrated in FIG. 3 and FIG. 4 can compromise the integrity platelet a vesicles, resulting in a high yield of desirable growth factors.

As shown in FIG. 3A, the lysis/activation chamber 104 and fluid pathway 105 of FIG. 1 may be implemented in part as a platelet-rich plasma chamber (PRP chamber 300) having any suitable volume. The PRP chamber 300 receives platelet-rich plasma, typically autologous PRP prepared from blood drawn from a patient. PRP may be input into the PRP chamber 300 through an input port 302 (FIG. 4, Step 402). Subsequently, after lysis/activation, PL solution may be withdrawn through an outlet port 304 and used as described herein for therapeutic purposes. The outlet port 304 includes a filter 306, for example a Polyethersulfone (PES) membrane filter, a Polyvinyl Difluoride (PVDF) filters or other syringe type filter that optimally features both low protein binding and possesses relatively high flow rates. The outlet port 304 may be placed in fluid communication with a vacuum and collection system 308 to facilitate PL solution collection.

As shown in FIG. 3B, the lysis/activation process may be initiated by filling the PRP chamber with PRP to a select level. A platelet count may be obtained prior to lysis/activation. Then, as shown in FIG. 3C, the PRP chamber may be rotated at a sufficiently high rate, centrifuge fashion, causing the platelets to collect on the outer wall of the PRP chamber 300 (FIG. 4, Step 404). A vacuum may then be applied to the outlet port causing withdraw of plasma (FIG. 3D). As shown in FIG. 3E-3F, the platelets remain in the chamber, either at or near the PRP wall, or captured by the filter 306. As the platelets collect at the filter interface, the vacuum cycle may be continued to partially dry or entirely desiccate the platelets at the filter and otherwise within the PRP chamber (FIG. 3G) (FIG. 4, Step 406).

Desiccation can wholly or partially cause lysis of the platelets. As shown in FIG. 3H, the wholly or partially dried platelets may be suspended in a suitable fluid, for example, sterile “water for injections” (WFI) (FIG. 4, Step 408). The suspended platelets may, after a period of time be subjected to a supplemental chemical lysis/activating agents, for example CaCl (FIG. 4, Step 410). The platelet count may be determined at various points in time. Any combination of platelet drying, suspension and lysis/activating agent addition may be employed to cause adequate platelet lysis/activation. Then, as shown in FIG. 3I, the PL solution may be withdrawn from the PRP chamber, placed into a suitable vessel such as a syringe and reinjected into a patient to accomplish therapeutic goals (FIG. 4, Step 412-414).

Platelets are composed of granules; primarily α-granules (and to some degree dense granules) which granules are pre-packaged during platelet biogenesis. According to current theory, once a platelet is made and released from the megakaryocyte, it contains all of the growth factors that is will ever contain; most of which are packaged within the foregoing granules. Further, not every platelet appears to have identical contents with regards to granule content. So, once a platelet is lysed and/or activated, it is beneficial to disrupt the integrity of the granules to maximize growth factor content in solution. For example, as described with respect to FIG. 3H, granule disruption may occur during the addition of CaCl₂ after the ‘filter-drying’ step in the FIG. 3 series. However, those granules that remain intact during the filtration process can be kept from passing into the produce collection vessel if the pore size is too small and thereby removing valuable therapeutic contents from the final injectable. Accordingly, one embodiment includes a filter pore size of greater than 0.22 μm, for example 0.45 μm.

In any of the embodiments described above, the forces required to move the platelet containing solution through the system may be generated according to several alternative means. For example, the platelet containing solution may be introduced into the system 100 with a syringe. As the syringe plunger is depressed the platelet containing solution is caused to flow into the fluid pathway 105 of the system. A selected protocol of device operation may be implemented to identify a specific volume of platelet containing solution to be added to the machine such that full depression of the plunger causes substantially all of the platelet containing solution to be located inside of the lysis/activation chamber. Once the lysis/activation operation is complete, an outlet valve 106 may be activated and the empty platelet containing solution syringe may be disconnected, providing for the PL solution to flow out of the thermal lysis/activation chamber. Other embodiments of flow motivation and control may include one or more peristaltic pumps or other pumps, adjustable volumes, sections utilizing drip flow or other types of gravity induced flow.

The foregoing device and method embodiments describe several distinct methods for lysing and activating platelets. The disclosed methods may be combined in any order. As detailed below, many of these methods result in greater than 90% lysis of the platelets in solution. For example, one combination of methods particularly well suited for lysis, activation and growth factor concentration is multiple freeze thaw steps followed by the platelets being centrifuged out of solution and exposed to a selected concentration of calcium chloride and water to impart osmotic stress.

### EXAMPLES

The following examples are provided for illustrative purposes only and are not intended to limit the scope of the invention. The samples examined in each of the examples detailed below were prepared from blood drawn from four blood donor subjects. The initial blood draw was separated via centrifugation and the platelet rich plasma portion of the separation was isolated and mixed well. The platelet rich plasma was then split into equal volumes to undergo different lysis/activation processes as detailed below. In each example, four parameters indicative of lysis and/or platelet activation were measured. The TGF-β and VEGF growth factor protein concentrations were compared using commercially available ELISA kits. The amount of platelet lysis was determined using flow cytometery with two positive platelet surface markers CD41 and CD61 to determine the quantity of platelets before and after the lysis/activation process. The amount of platelet activation was determined with flow cytometery using an activated platelet surface marker CD62p to determine the number of platelets remaining after lysis that had been activated.

### Example 1

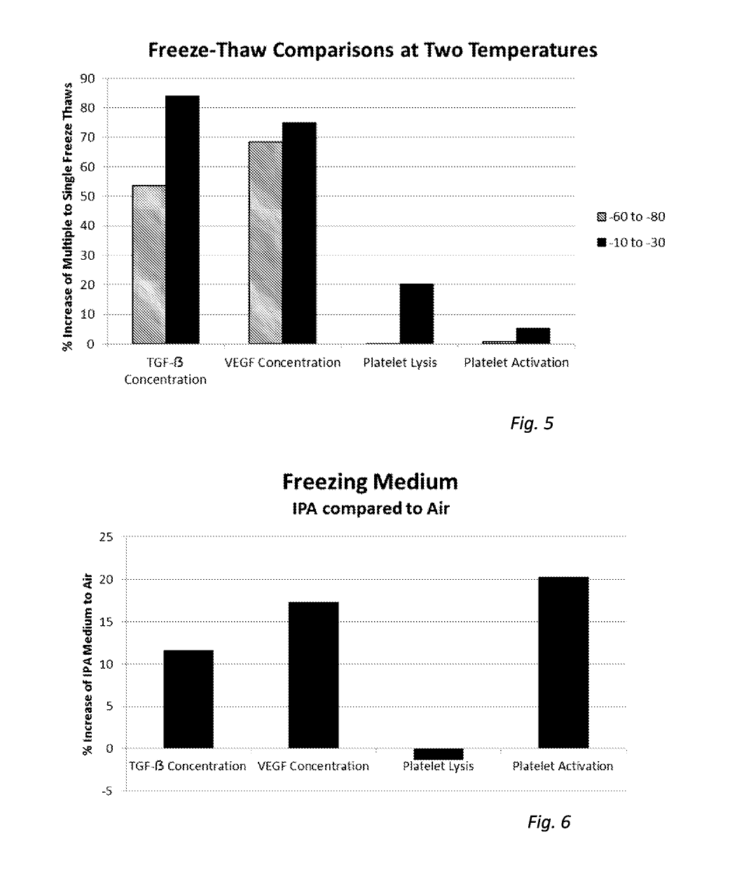

As noted above, multiple freeze thaw cycles can be utilized to cause the lysis or activation of platelets in solution and to promote exocytosis of therapeutic contents from the platelets. FIG. 5 graphically illustrates the percentage increase of platelet lysis, activated platelets remaining in solution and growth factor concentration between one freeze thaw cycle and at least three freeze thaw cycles. Similar tests were performed at two different freezing temperatures. The samples examined in Example 1 were prepared as described above. As illustrated in FIG. 5, an increase in the observed lysis/activation parameters was noted after multiple freeze/thaw cycles. In particular, multiple freeze/thaw cycles resulted in a large percent increase of TGF-β and VEGF growth factors detected when the tests were performed at either temperature range. An increase in platelet lysis and activation was also observed for multiple freeze/thaw cycles with the exception of the temperature range of −60° C. to −80° C.

### Example 2

Alternative freezing and thawing methods have been determined to impact the amount of observed lysis and activation. For example, FIG. 6 graphically represents the percentage increase in detected growth factor content, platelet lysis and platelet activation determined after using isopropyl alcohol (IPA) as a freezing medium with respect to using air as a freezing medium. The samples examined in Example 2 were initially prepared as noted above. Each sample was subjected to one freeze/thaw cycle. The platelet solutions tested using IPA as a cooling medium was subjected to a cold temperature in the range of −10° C. to −30° C. with a chilled IPA bath and thawed at a temperature in the range of 20° C. to 50° C. The platelet solutions examined using air as a cooling medium was placed in a circulating air freezer at a temperature of −60° C. to −80° C. and thawed in air at a temperature of 20° C. to 50° C. It may be observed from FIG. 6 that the use of isopropyl alcohol as a freezing medium results in a modified platelet solution with a higher growth factor concentration, a small reduction in lysis, but an increase in activated platelets when compared to the modified platelet solution prepared with compressed air as a cooling medium. Example 2 demonstrates that platelet activation can play a significant role in the release of therapeutic platelet contents in the presence of a lesser degree of platelet lysis.

### Example 3

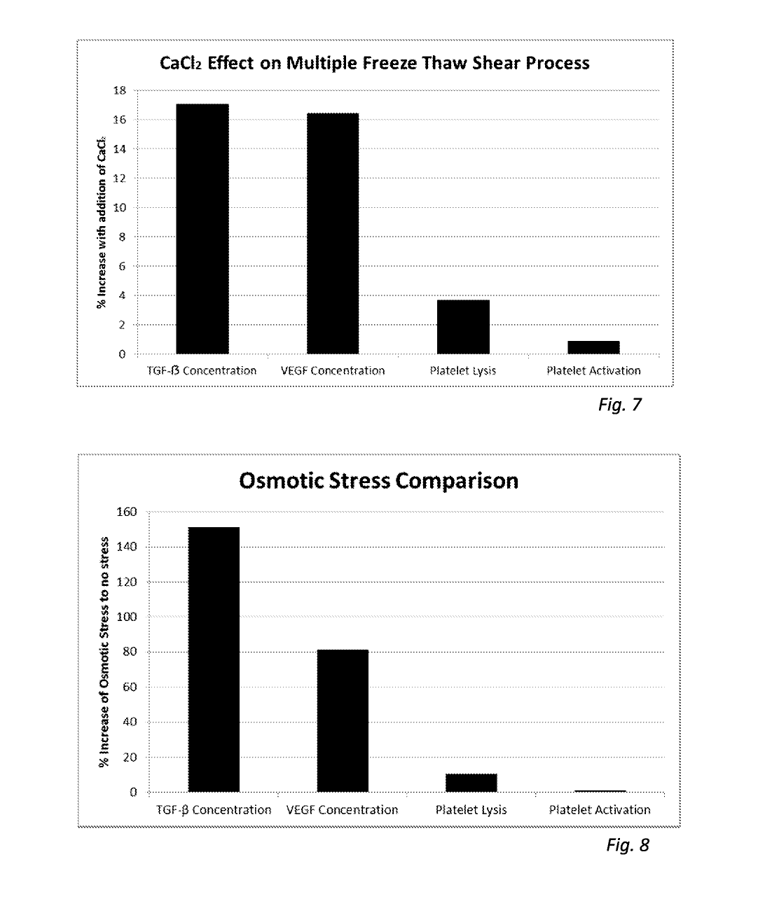

FIG. 7 graphically illustrates the percentage increase of growth factor concentration, platelet lysis and platelet activation with the addition of a lysis/activation agent, CaCl₂ as one step of the lysis/activation process. The samples examined in example 3 were initially prepared as described above. Each sample was then subjected to multiple freeze/thaw cycles with each thaw step followed by a shear processes in which the platelet solution was forced through a 27 g needle at least 5 times. Selected samples then were subjected to the addition of 0.5 to 4 μM CaCl₂. FIG. 7 illustrates the percentage increase of the observed lysis/activation parameters for samples subjected to a final CaCl₂ addition compared to samples prepared with the same freeze/thaw and shear stress steps but with no final CaCl₂ addition.

### Example 4

FIG. 8 graphically illustrates the percentage increase of growth factor concentration, platelet lysis and platelet activation of a sample undergoing osmotic stress with a hypotonic solution compared to a sample not subjected to osmotic stress. The samples examined in FIG. 8 were initially prepared as described above. Certain samples were then processed using a centrifuge to remove platelets from the platelet rich plasma solution. The removed platelets were then suspended in hypotonic water subjecting the platelets to osmotic stress. The control samples were not subjected to osmotic stress. The growth factor concentrations of the platelet solution subjected to an osmotic stress is significantly higher than the growth factor concentrations of samples not subjected to osmotic stress.

### Example 5

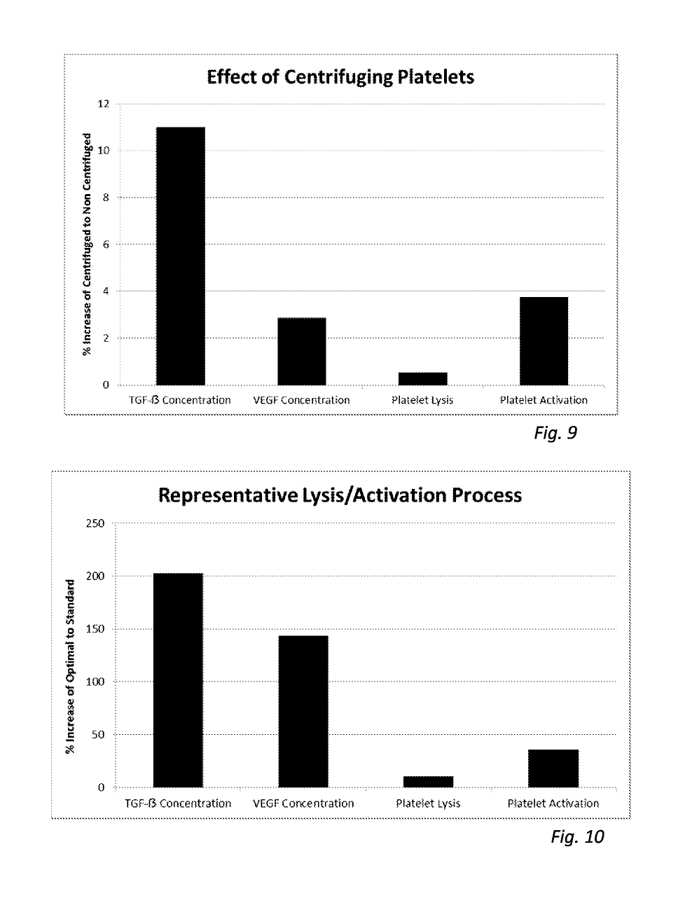

Example 5 illustrates the results of a test where platelets were visibly centrifuged out of solution, the supernatant decanted and the resulting pelleted platelets subjected to freezing at −10° C. to −30° C. The platelet pellet was then re-suspended in the previously withdrawn supernatant before being analyzed. The control group of example 5 was subjected to one freeze/thaw cycle at similar temperatures while remaining in solution. A comparison of the results of a lysis/activation analysis performed on each group are graphically illustrated in FIG. 9. The growth factor concentration, platelet lysis and platelet activation are observed to be somewhat greater when the platelets are processed with a freeze/thaw cycle after being removed from solution using a centrifuge when compared to similarly processed platelets left in solution.

### Example 6

FIG. 10 graphically illustrates the percentage increase of growth factor concentration, platelet lysis and platelet activation for a representative combination of lysis and activation methods. One group of the test platelets of Example 6 were visibly centrifuged out of solution. The container housing the resulting platelet pellet was placed in an IPA medium at −10° C. to −30° C. for a duration of between 5 and 60 minutes. The platelets were then re-suspended in a hypotonic solution and allowed to lyse/activate for 1 to 20 minutes at room temperature. CaCl₂ at a concentration range of 0.5 to 4 μM was then added to the solution and the sample placed back in the earlier withdrawn supernatant for another freeze cycle. The sample was then thawed at a temperature of between 20° C. and 50° C. and analyzed. The control samples were subjected to a single freeze/thaw cycle in a freezer with air as the circulating medium. A very large increase in growth factor concentration and activation is noted after the combination of steps noted above. The percentage increase of platelet lysis was observed to be only 10% however. Therefore it may be concluded that the single freeze/thaw procedure performed in solution accomplished the lysis of approximately 90% of the platelets whereas the combination method resulted in the lysis of approximately 99.8% of the platelets. The percentage increase of activated platelets remaining in solution was approximately 36% in the combination method compared to the simple single freeze/thaw method.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

What is claimed is:

1. A device comprising:
a housing;
an input port into the housing providing for the input of a platelet containing solution;
a lysis/activation chamber in fluid communication with the input port and positioned within the housing, wherein one or more platelets within the platelet containing solution input to the device at the input port are caused to undergo lysis or activation in the lysis/activation chamber, thereby creating modified solution; and
an outlet port from the housing, in fluid communication with the lysis/activation chamber, providing for the modified solution to be removed through the outlet port.

2. The device of claim 1 further comprising a heating and cooling module in thermal communication with the lysis/activation chamber, said heating and cooling module providing for the thermal lysis or activation of one or more platelets of the platelet containing solution within the lysis/activation chamber.

3. The device of claim 2 wherein the lysis/activation chamber comprises a length of disposable sterile tubing.

4. The device of claim 2 wherein the heating and cooling module causes the platelet containing solution to wholly or partially freeze and wholly or partially thaw within the lysis/activation chamber.

5. The device of claim 4 wherein the heating and cooling module causes the platelet containing solution to undergo more than one freezing and thawing cycle within the lysis/activation chamber.

6. The device of claim 1 further comprising at least one supplemental input port into the housing and in communication with the lysis/activation chamber, said supplemental input port providing for the introduction of a substance into contact with the platelet containing solution to cause the lysis or activation of one or more platelets within the lysis/activation chamber.

7. The device of claim 1 further comprising at least one acoustic transducer within the housing, the acoustic transducer being mechanically coupled to the lysis/activation chamber, said acoustic transducer being configured to communicate acoustic energy to the lysis/activation chamber to cause sonic lysis or activation of one or more platelets within platelet containing solution in the lysis/activation chamber.

8. The device of claim 1 wherein the lysis/activation chamber further comprises a filter operatively associated with the outlet port providing for the removal of a fluid from the outlet port while maintaining platelets within the lysis/activation chamber.

9. The device of claim 8 wherein the filter comprises a PVDF or PES filter having a filter pore size of less than or equal to 0.45 μm.

10. The device of claim 8 wherein the filter comprises a PVDF or PES filter having a filter pore size of greater than or equal to 0.22 μm.

11. The device of claim 8 further comprising a vacuum system within the housing, the vacuum system being operatively associated with the outlet port and providing for withdraw of plasma from the platelet containing solution.

12. The device of claim 8 further comprising a rotation device coupled to the lysis/activation chamber and providing for the rotation of the lysis/activation chamber within the housing.

13. A method comprising:
introducing a platelet containing solution into the input port of a device having a housing;
flowing the platelet containing solution from the input port to a lysis/activation chamber within the housing;
causing the lysis or activation of one or more platelet within the platelet containing solution in the lysis/activation chamber to create a modified solution; and
collecting the modified solution through an outlet port of the housing.

14. The method of claim 13 further comprising reinjection of the modified solution into a patient.

15. The method of claim 13 wherein platelet lysis or activation is caused within the lysis/activation chamber by cyclically cooling and heating the platelet containing solution in the lysis/activation chamber causing one or more partial or complete freeze and thaw cycles within the platelet containing solution.

16. The method of claim 13 wherein platelet lysis or activation is caused by mixing the platelet containing solution with a lysis or activation causing agent in the lysis/activation chamber.

17. The method of claim 13 wherein platelet lysis or activation is caused by applying acoustic energy to the platelet containing solution in the lysis/activation chamber.

18. The method of claim 13 wherein platelet lysis or activation is caused by subjecting the platelet containing solution to osmotic pressure in the lysis/activation chamber.

19. The method of claim 13 wherein platelet lysis/activation is caused by partially or wholly drying the platelet containing solution in the lysis/activation chamber.

20. The method of claim 19 wherein platelet lysis/activation is further caused by mixing the dried platelet containing solution with a lysis or activation causing agent in the lysis/activation chamber.

21. The method of claim 19 further comprising filtering plasma from the platelet containing solution prior to partially or wholly drying the platelet containing solution in the lysis/activation chamber.

22. The method of claim 19 further comprising concentrating the platelets in the platelet containing solution prior to partially or wholly drying the platelet containing solution in the lysis/activation chamber.

23. The method of claim 22 wherein the platelets are concentrated by rotating the lysis/activation chamber around an axis.

24. The method of 13 further comprising disposing of at least one device component wetted by one of the platelet containing solution or the modified solution after a single use.
